(19) United States
(12) Patent Application Publication
(10) Pub. No.: US 2016/0200984 A1
(43) Pub. Date: Jul. 14, 2016

(54) DESORPTION OF BITUMEN FROM CLAY PARTICLES AND MATURE FINE TAILINGS

(71) Applicant: GOVERNORS OF THE UNIVERSITY OF ALBERTA, Edmonton (CA)

(72) Inventors: Christian DETELLIER, La Peche (CA); Sadok LATAIEF, Gatineau (CA); Jonathan FAFARD, Gatineau (CA); Gustave Kenne DEDZO, Gatineau (CA)

(21) Appl. No.: 15/076,019

(22) Filed: Mar. 21, 2016

Related U.S. Application Data
(62) Division of application No. 14/083,824, filed on Nov. 19, 2013.

(51) Int. Cl. C10G 1/04; G01N 33/28

(52) U.S. Cl. CPC C10G 1/045; G01N 33/2823

(57) ABSTRACT

A method for desorption of bitumen from clay particles. Also a method for the desorption of asphaltenes from clay particles and a method for the desorption of bituminous fractions from mature fine tails (MFT). The method for desorption of bitumen from clay particles involves interacting, in a suitable organic solvent, a clay-bitumen composite with a compound capable of stabilizing the bitumen in the organic solvent and adsorbing to clay particles such that the compound replaces the bitumen in the clay-bitumen composite. A substantial amount of the compound that is capable of stabilizing the bitumen in the organic solvent is recovered with the clay particles, while the bitumen is released. Similarly, the method for desorption of asphaltenes from clay particles also involves interacting a clay-asphaltene composite with a compound capable of stabilizing the asphaltenes in an organic solvent, recovering the compound with the clay particles, and releasing the asphaltenes into solution.

### CROSS REFERENCE TO RELATED APPLICATION

This application is a divisional of U.S. Provisional Patent Application Ser. No. 14/083,824 filed Nov. 19, 2013, which is incorporated herein by reference.

### FIELD

The disclosure relates to the separation of bitumen, asphaltenes and bituminous fractions from clay particles, oil sands, and mature fine tails.

### BACKGROUND

With the demand for crude oil at its current level, the recovery of hydrocarbons from oil sands is of interest. Oil sands are so-called unconventional hydrocarbon reserves. Oil sands contain mixtures of silicates, clay, water and hydrocarbon compounds such as bitumen. Processing of oil sands involves the extraction or separation of hydrocarbon compounds from the remaining fractions of the oil sand. Bitumen is a hydrocarbon compound present in oil sands that is of particular interest. There are different methods to extract bitumen from oil sands. For example, aqueous-based methods, solvent extraction, supercritical fluid extraction, use of microemulsions, etc. have been used to separate bitumen from oil sands. Factors such as ore type, surfactants used, time, temperature, pH, mixing intensity, inorganic ions and viscosity of bitumen can influence the extraction of bitumen from oil sands.

Different oil sands have different compositions, but generally, most oil sands consist of mixtures of bitumen, quartz, sands, clays, trace minerals and water. The amount and composition of the clays varies with the type of oil sand. The clay minerals in oil sands include chlorite, kaolinite, kaolinite-smectite, illite and illite-smectite, with kaolinite and illite typically being the most abundant. Kaolinite is known to have siloxane and aluminol surfaces. Bitumen and other hydrocarbons adhere to the surfaces of these clay minerals.

Mature Fine Tails (MFTs) are materials left over after the process of separating the bituminous fraction from oil sands. They are a gel-like material resulting from the clay fines contained within the oil sands, which material cannot be separated in the previous extraction steps. Bitumen strongly adsorbs onto the MFTs. MFTs represent a major environmental problem.

Bitumen is composed of a number of components, including asphaltenes. Asphaltenes have complex structures comprising sulfur, nitrogen, aromatic and naphthenic groups. Asphaltenes and other crude oil components with polar functionality can adsorb onto clay mineral surfaces present in oil sands. Asphaltenes are recognized to be the most difficult fraction of bitumen to remove from the oil sands. Extraction of bitumen from oil sands is difficult because of the presence of fine particles of clay minerals in the oil sands makes it difficult to separate the bitumen from these components. Maximizing the recovery of bitumen from a given oil sand would minimize environmental damage associated with hydrocarbon recovery from oil sands.

### SUMMARY

A method for separation of bitumen and asphaltenes from clay particles in non-aqueous solvents is disclosed. A method of separation of bitumen from mature fine tailings is also disclosed. The method involves contacting a solution comprising a bitumen-clay composite or an asphaltene-clay composite with a compound that is capable of disrupting the interaction of the bitumen and the asphaltenes with the clay particles in the presence of an organic solvent. The compound is able to stabilize heavy fraction of bitumen in solution, and to adhere or adsorb onto the mineral surfaces of the clay particles. In so doing, the compound disrupts the interaction between the bitumen and the clay particles. The clay particles, with bound adsorbent, can be isolated away from the bitumen, and the bitumen is released into solution. Desorption can be carried out in organic solvents, such as toluene. This separation is also known as a desorption process because bitumen is desorbed from the clay particles. The compound capable of disrupting the interaction is known as an adsorbent. The method can be used to recover residual bitumen from mature fine tailings (MFTs), and to recover bitumen from oil sands. The method constitutes an environmentally friendly way to extract bitumen from oil sands.

### BRIEF DESCRIPTION OF THE DRAWINGS

FIG. 1a is a chart showing the desorption percentage of C5-asphaltenes from kaolinite as a function of increased amount of cellulose.

FIG. 1b is a graph showing the amount of cellulose recovered with the extraction residue after desorption of C5-asphaltenes in presence of cellulose.

FIG. 2a is a chart showing the desorption percentage of C5-asphaltenes from kaolinite as a function of increased amount of methylcellulose (MC), hydroxypropylcellulose (HPC) and ethylcellulose (EC).

FIG. 2b is a graph showing the amount of MC recovered with the extraction residue after desorption of C5-asphaltenes in presence of MC.

FIG. 2c is a graph showing the amount of HPC recovered with the extraction residue after desorption of C5-asphaltenesin presence of HPC.

FIG. 2d is a graph showing the amount of EC recovered with the extraction residue after desorption of the C5-asphaltenes in presence of EC.

FIG. 3a is a chart showing the desorption percentage of C5-asphaltenes from kaolinite as function of increased amount of polyethylene glycol (PEG₁₀₀₀).

FIG. 3b is a graph showing the amount of PEG₁₀₀₀ recovered with the extraction residue after desorption of C5-asphaltenes in presence of PEG₁₀₀₀.

FIG. 4 is a graph showing the variation of desorption percentage of C5 asphaltenes as a function of the amount of Kaolinite-C5 asphaltene composite. The desorption medium is toluene (T), toluene with PEG (T-PEG) or toluene with EC (T-EC).

FIG. 5 is a graph showing the desorption percentage of C5 asphaltenes when different adjuvants were used.

FIG. 6a is a chart showing a comparison of desorption percentage of C5-asphaltenes in Toluene and water saturated toluene in the presence of EC or PEG₁₀₀₀.

FIG. 6b is a chart showing a comparison of desorption percentage of C5-asphaltenes in Toluene and water saturated toluene in the presence of EC or PEG₁₀₀₀, when successive desorptions are carried out.

FIG. 6c is a schematic representation of the mechanism of desorption of asphaltenes from a kaolinite composite in the presence of PEG or EC in toluene.

FIG. 7a is a XRD pattern of mature fine tailings (MFTs), highlighting the presence of major minerals.

FIG. 7b is a ¹³C NMR spectrum of mature fine tailings (MFTs).

FIG. 7c is a chart showing desorption percentage of bitumen from MFTs in toluene- or water-saturated toluene in presence of EC or PEG₁₀₀₀ as adsorbent.

FIG. 8 is a chart showing desorption percentage of bitumen from MFTs in toluene (T), methyl tert-butyl ether (MTBE), anisole (AN) or 2-methylfuran (2-MF) in the presence of EC or PEG₁₀₀₀ as adsorbent.

### DETAILED DESCRIPTION

Crude oil contains a variety of compounds. The highest concentration of polar organic compounds generally is found in the heavy ends (e.g. the high molecular weight components) of crudes, particularly in the asphaltenes and resin fractions. Because of the polar nature of asphaltenes and because of their high molecular weight, asphaltenes adsorb onto clay surfaces. In practice, when considering a crude oil, the extent of the adsorption of asphaltenes onto the clay surface may depend on factors such as (1) the presence, thickness, and stability of water films on the clay mineral surface; (2) the chemical and structural nature of the clay mineral substrate; (3) the asphaltenes and resins content of the crude; (4) the presence of asphaltenes and resins in crude oils in the form of colloidal micelles or aggregates; and (5) the ability of the hydrocarbon fraction of the crude to stabilize these colloidal aggregates in the oil or even to dissolve them into true solution. These factors are relevant when considering how to disrupt these interactions and thereby separate the asphaltenes from the clay particles. The presence of a compound that could stabilize asphaltenes in solution (e.g. a stabilizer) could favorably affect the recovery of hydrocarbons from the clay surfaces. For example, polymers such as ethyl cellulose or PEG which are soluble in organic solvents such as toluene could be used to stabilize the asphaltene fraction.

Different oil sands have different compositions, but generally, most oil sands consist of mixtures of bitumen, quartz, sands, clays, trace minerals and water. It should be possible to extract hydrocarbons such as bitumen from other components of oil sands by disrupting the chemical interactions between the bitumen and the clay mineral surfaces. It should also be possible to disrupt the interaction between asphaltenes and clay particles. In other words, the dual effects of:

a) the chemical affinity of clay particles to various compounds (e.g. adjuvants) and b) the ability of various compounds to stabilize asphaltenes can be exploited. Compounds that favor the solubilization of asphaltenes and absorb physically onto the surface of the clay particles can cause the clay particles to be segregated away from the bitumen or asphaltenes that had been associated with the clay particles. The addition of an adjuvant to an asphaltene/clay mineral dispersed in solution can result in the adsorbent becoming partially adsorbed onto the clay particle. The residue after desorption can then be recovered by centrifugation or by sedimentation and contain clay particles on which residual bitumen and adjuvant are adsorbed. Desorbed bitumen can then be easily recovered in the supernatant by distillation of the solvent. The solvent can then be recycled and reused.

The present disclosure is based on the recognition that the chemical properties of clay particles can be used in a method and system to separate asphaltenes and bitumen from the clay particles. The method relates to the recovery of bitumen from oil sands in a non-aqueous solvent. The method exploits the ability of certain organic compounds (e.g. also referred to herein as adjuvants) to stabilize the heavy fraction of bitumen in the organic solution and also to act as competitive adsorbents with respect to the heavy fraction of bitumen, thereby facilitating the extraction of bitumen from the oil sands.

Typical bitumen extraction processes utilize large quantities of water. The water can end up in tailings ponds, where it has to be tested for contaminants. In the present method, an organic solvent is used instead of water. The replacement of water with an organic solvent avoids the need to use a scarce resource such as water, and avoids the build-up of pools of contaminated water.

As noted above, mature find tailings (MFTs) are materials left over after the process of separating the bituminous fraction from oil sands. They are a gel-like material resulting from the clay fines contained within the oil sands which cannot be separated in the previous extraction steps. Bitumen strongly adsorbs onto the MFTs. MFTs represent a major environmental problem. The present disclosure provides an efficient way to separate bitumen from MFTs and could be applied to oil sands.

The present disclosure relates to a method of separating bitumen and components of bitumen, such as asphaltenes, from clay particles and from MFTs using a non-aqueous based extraction method. The method is based on two synergetic effects achieved when a suitable adsorbent is added to the system: (i) the adsorption of the adsorbent (herein referred as a competitive adsorbent, an adsorbent or an adjuvant) onto the clay particles; and (ii) the stabilization of heavy fractions of bitumen or asphaltenes in the organic solvent by the adsorbent (herein referred as the stabilizing function). Thus, the same compound acts both as a competitive adsorbent and as a stabilizer. Examples of compounds or adsorbents that can be used for the competitive desorption process disclosed herein include polyethylene glycol (PEG), cellulose, various cellulose derivatives and various other compounds with similar structural properties.

A typical component in clay particles found in oil sands is kaolinite. The strong interactions of kaolinite with bitumen generally and with the asphaltenes fraction of bitumen in particular are well-established. Thus, minerals such as kaolinite are suitable models for mimicking the interaction between clay particles and bitumen in oil sands. Kaolinite occurs as micron-sized platelets with a strong tendency for aggregation, forming a book-like structure. The platelets display siloxane and aluminol surfaces onto which an adsorbent can adhere. The present method of adsorbing an adsorbent onto the clay particle may be carried out in non-aqueous solvents, such as toluene. While many adsorbents could be used in the present method, it may be beneficial to carry out the method using abundant, inexpensive and environmentally-acceptable adsorbents. For example, adsorbents such as cellulose and cellulose derivatives may be used. Polyethylene glycol and similar compounds may also be used. It is useful if the competitive adsorbent used is soluble in the organic solvent in which the desorption is carried out.

The present method involves separating bitumen or components of bitumen such as asphaltenes from clay particles or from the mineral surfaces of clay particles (the mineral surface includes minerals such as kaolinite). A compound or adsorbent is selected which has a high affinity for the kaolinite and that can also stabilize asphaltenes in solution. The compound is added to a kaolinite-asphaltenes composite or to a clay-bitumen composite, for example. The mixture is then agitated to improve the contact between the different constituents of the system. The adsorbent and clay particle become bound together (and can be collected as the residue fraction), while the asphaltenes or bitumen is released into the solution.

When the method involves separation of asphaltenes from clay particles, the asphaltenes can be quantified using UV-Vis absorption spectra to monitor the release of asphaltenes from the clay particles as adsorbent is added. Asphaltenes can also be qualitatively analyzed using ¹³C MAS-NMR. Thermal gravimetric analysis (TGA) may be used to determine the amount of adsorbent recovered in the residue after desorption.

### Suitable Adsorbents

The additives used should ideally possess various characteristics, including: (i) good solubility in the solvent; (ii) the ability to form strong hydrogen bonds with the surfaces of kaolinite or clay minerals; (iii) low environmental impact (as substantial amount will remain on solid residue after extraction); (iv) good activity when used in small amounts in the system; and (v) low cost. Natural or modified cellulosic polymers provide good results, these polymers can provide a very dense network of hydrogen bonds, thus favoring adsorption onto the clay surfaces of the oil sands or mature fine tailings (MFTs), as will be discussed in the Examples. Suitable adsorbents can include methylcellulose (MC), ethylcellulose (EC) and hydroxypropyl cellulose (HPC), or mixtures thereof. Polyethylene glycol (PEG) may also be used. The chemical structures of these compounds are shown below. Generally, suitable adsorbents have at least some polar constituents to allow for binding to the clay particles (or the mineral surfaces of the clay particles). Other suitable adsorbents may include alcohols, fatty acids or diols, for example.

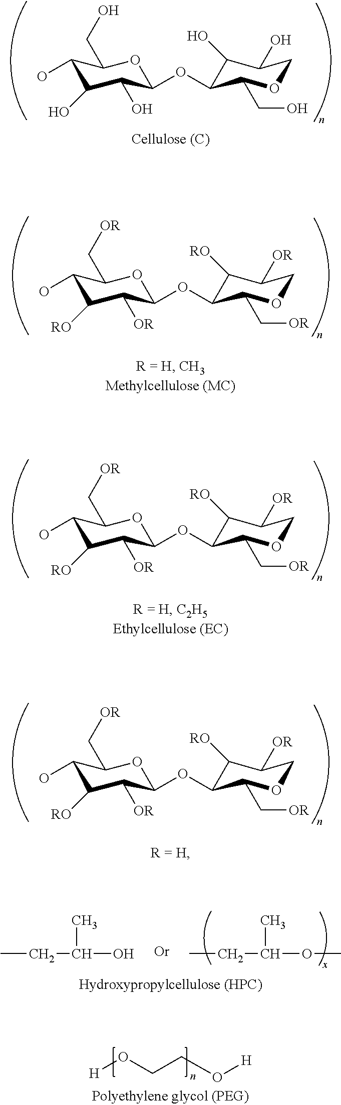

### Method

The method involves dispersing a clay-bitumen composite (synthetic or natural) in a solvent and adding an adjuvant (noted here competitive adsorbent or stabilizer). The clay-bitumen composite may be a kaolinite-C5 asphaltenes composite or a mixture of mature fine tails (MFTs). The mixture is then stirred at ambient temperature to promote desorption of bitumen from the clay, favored by the combined effects of solvent and adjuvant. The amount of bitumen desorbed is determined after the centrifugation of the mixture, by the quantitative analysis of the supernatant.

The amount of asphaltenes recovered following desorption can also be quantified using other spectroscopic techniques, for example. Asphaltenes can be quantified using the absorbance at 450 nm. Calibration curves can be prepared showing absorption as a function of asphaltene concentration, and these curves can be used to quantify the efficiency of the desorption process. The amount of residual bitumen recovered from the MFTs can be quantified by a similar method.

### Suitable Solvents

The ideal solvent should possess various properties including: (i) the ability to solubilize asphaltenes. Asphaltenes are known to be poorly soluble because of their tendency to form aggregates. Indeed, solvents that cannot dissolve asphaltenes, such as linear saturated hydrocarbons, show very low bitumen desorption. This can be explained by the strength of Tr-Tr interactions between these compounds and their strong interactions with clays surfaces. However, aromatic solvents such as toluene can solubilize asphaltenes by breaking down or reducing the strength of these interactions; (ii) the ability to solubilize the adjuvant to overcome mass transport issues in the system and to promote contact between the competitive adsorbent and clay-bitumen composite; (iii) a low boiling point (between 60° C. and 90° C.), so that at the end of the process, the solvent could be easily recovered by distillation.

Aromatic solvents such as toluene have the ability to dissolve asphaltenes. Some adjuvants tested are soluble in aromatic solvents (e.g. PEG and EC are soluble in toluene). While some solvents may not be optimal to carry out the method, parameters such as time for the adsorption and temperature at which the adsorption reaction is carried out may be increased to improve the desorption of bitumen and/or asphaltenes from the clay particles.

### Suitable Temperature Conditions

The present method may be carried out at a variety of temperatures. The Examples discussed below were carried out at temperatures ranging from 22-25° C. Increasing temperature may improve performance and decreasing temperature may negatively affect the competitive adsorption.

### EXAMPLES

### Example 1

Asphaltenes represent the fraction of bitumen presenting the strongest adsorption on clay surfaces and especially on the kaolinite surfaces. A kaolinite-C5 asphaltenes composite (KAC) was prepared as follows: Kaolinite (5 g) was dispersed in 500 mL of 1 g L⁻¹ asphaltenes solution prepared in toluene. After stirring for 12 hours at ambient temperature, the solid was collected by centrifugation, dried in an oven and kept in a sealed vial. The amount of adsorbed asphaltenes was determined by means of UV-visible spectrophotometry at 450 nm. Composites used throughout had an asphaltenes content of 39 mg g⁻¹. Toluene (99.9%) was purchased from Fisher, kaolinite (KGa-1 b, Ga.) obtained from the Source Clays Repository of the Clay Minerals Society (Purdue University, West Lafayette, Ind., USA). C5 asphaltenes were obtained from Syncrude Canada Ltd.

A series of compounds were tested to identify the adjuvant that could provide desorption of asphaltenes from kaolinite. Cellulose was chosen because of the high density of hydroxyl functions that would provide strong hydrogen bond interactions in the system and also because of its abundance as a biopolymer. The cellulose used was amorphous cellulose purchased from Aldrich.

In these experiments, 10 mg of KAC (39 mg/g) was introduced in a vial with one amount of cellulose as competitive adsorbent. 10 mL of toluene was then added and the mixture stirred for 12 h at ambient temperature. After centrifugation, the amount of asphaltenes released in solution was determined at 450 nm. Cellulose mass percentage relative to KAC was varied from 0 to 200.

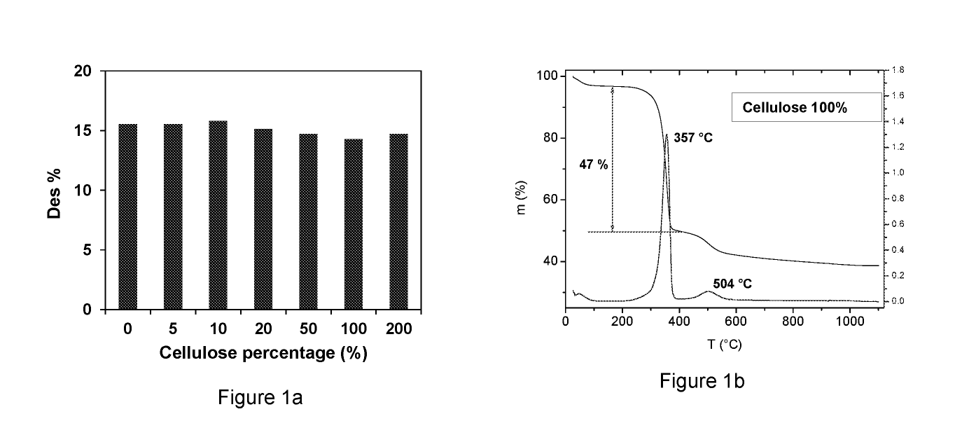

The results in FIG. 1 a show that the presence of low amounts of cellulose in the system only moderately improves the desorption of asphaltenes. At higher amounts of cellulose, desorption is not favoured. This can be explained by the non-solubility of cellulose in toluene which limits the interactions with organoclays because of mass transport issues.

FIG. 1b presents the TGA (thermogravimetric analysis) of the dry residue after the experiments. This figure shows that cellulose is recovered entirely in the residue. SEM images of KAC and SEM images of the residue after desorption in the presence of cellulose were obtained. These showed that cellulose interacts strongly with the kaolinite platelets, promoting their disaggregation. This property can be used to increase the efficiency of the bitumen removal in the presence of other adjuvants.

### Example 2 (Cellulose Derivative)

In another series of experiments, the functionalized cellulose derivatives methylcellulose (MC), hydroxypropylcellulose (HPC) and ethylcellulose (EC) were tested. These polymers may provide a better interaction with the solvent compared with cellulose. MC, HPC and EC (48% ethoxyl) were purchased from Aldrich.

To conduct the experiments, 10 mg of KAC (39 mg/g) was introduced in a vial with one amount of cellulose derivative as competitive adsorbent. 10 mL of toluene was then added and the mixture stirred for 12 h at ambient temperature. After centrifugation, the amount of asphaltenes released in solution were determined at 450 nm. Cellulose derivative mass percentage relative to KAC varied from 0 to 200.

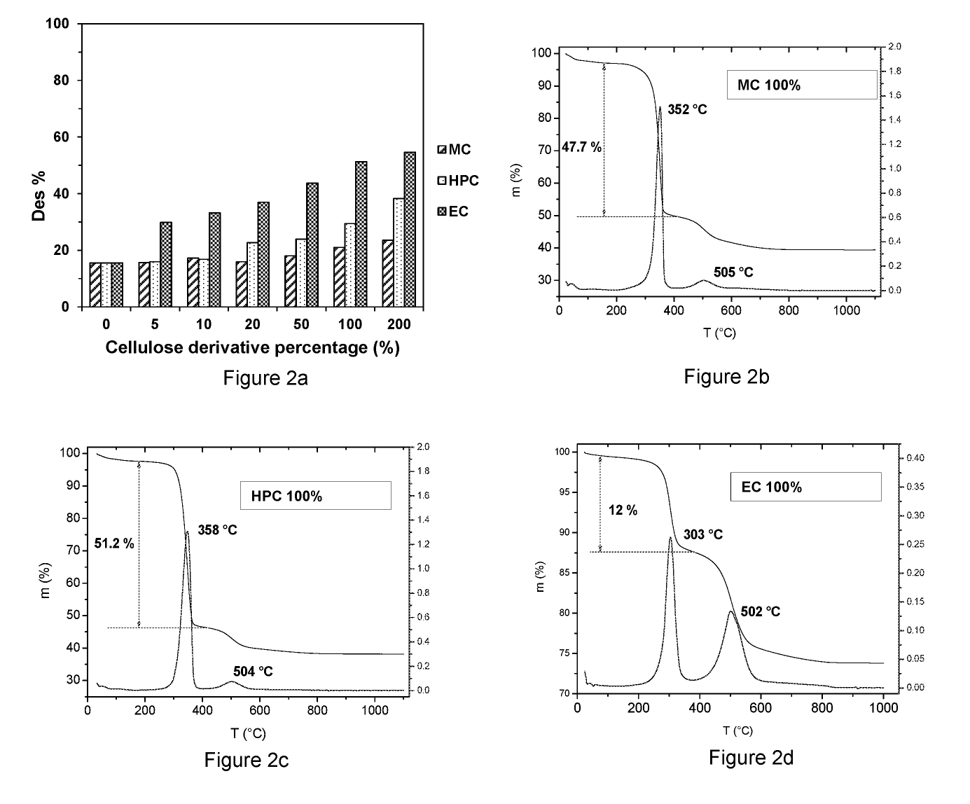

The results in FIG. 2 (a) show that MC had almost no effect, HPC promoted asphaltenes desorption when present at a high percentage (more than 20%). On the contrary, EC show good behaviour even at 5%. At 200%, more than the half of organic matter was removed. These results can be correlated to the solubility of these compounds as shown by TGA of the dry residues presented in FIG. 2 (b-d). MC and HPC were recovered completely with the solid, indicating their poor solubility in toluene. However, the performance of HPC likely relates to the strong interaction of hydroxypropyl functions with toluene, inducing the dispersion of the polymer in solution. When EC was used, only 25% of the polymer is recovered. This was likely adsorbed on clay surface since EC is soluble in toluene.

### Example 3 (PEG)

The nature of the action of competitive adsorbents was investigated to determine whether desorption is due to interactions with kaolinite or asphaltenes. Polyethylene glycol (PEG) is soluble in toluene (e.g. low molecular weight PEG is soluble in toluene). The chemical structure of this polymer allows interactions both with kaolinite and asphaltenes. PEG₁₀₀₀ was purchased from Aldrich. Throughout this document, unless otherwise specified, the term PEG is used to refer to PEG₁₀₀₀ where 1000 is the average molecular weight (g/mol).

10 mg of KAC (39 mg/g) was introduced in a vial with one amount of PEG₁₀₀₀ as competitive adsorbent. 10 mL of toluene was then added and the mixture stirred for 12 h at ambient temperature. After centrifugation, the amount of asphaltenes released in solution was determined at 450 nm. PEG mass percentage relative to KAC was varied from 0 to 200.

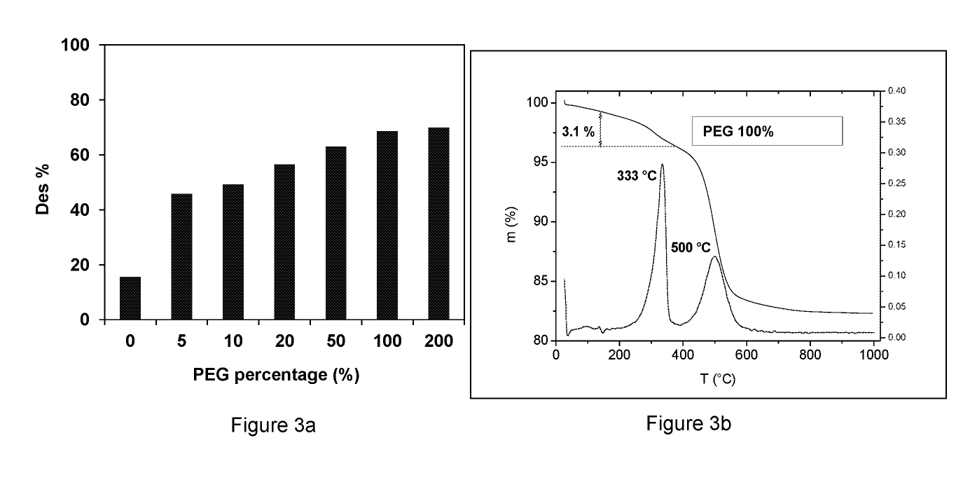

The desorption percentages recorded as a function of the amount of polymer in the medium (FIG. 3a) showed that only 5% of PEG was enough to remove 45% of adsorbed asphaltenes (three times the value obtained with toluene alone). This performance was improved by increasing the amount of PEG used to reach a value of 70% when 200% of PEG is used. TGA of the dry residue (FIG. 3b) revealed that only 6% of PEG is recovered with the solid. This confirmed that the polymer present in solution favored asphaltenes recovery and also adsorption onto the clay surfaces. In this case, the PEG (which is more soluble than EC), was more efficient than EC.

### Example 4 (Effect of the Amount of Solvent)

To enhance desorption, the influence of the volume of toluene used for desorption was studied. In theory, a large volume of solvent should promote desorption through better dispersion of the constituents used in the system. Only EC and PEG were used because of their efficiency.

The procedure was identical to that described in Example 1 except that the amounts of KAC added in 10 ml of toluene were varied so that the concentrations of KAC were between 0.1 and 5 g/L. 100% (by weight) of adjuvant (determined with respect to the mass of KAC used).

Typically, various amounts of KAC (39 mg/g) ranging from 1 mg to 50 mg (0.1 to 5 g/L) were introduced in vials with specific amounts of EC or PEG₁₀₀₀ as adjuvants. 10 mL of toluene was then added and the mixture stirred for 12 h at ambient temperature. After centrifugation, the amount of asphaltenes released in solution was determined at 450 nm. EC or PEG₁₀₀₀ mass percentage relative to KAC was maintained at 100% for all experiments.

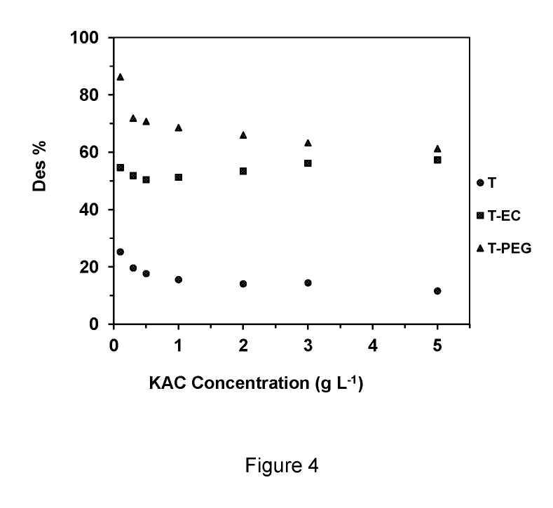

As shown in FIG. 4, high amounts of solvent tended to increase the desorption percentage slightly. High amounts of solvent are not necessary to obtain good desorption efficiencies.

### Example 5 (Other Adjuvants)

A series of other compounds were tested as adjuvant or adsorbent. The operating conditions were similar to those described above: 10 mg of KAC (39 mg/g) was introduced in a vial with equal amount of competitive adsorbent, 10 mL of toluene was then added and the mixture stirred for 12 h at ambient temperature. After centrifugation, the amount of asphaltenes released in solution was determined at 450 nm.

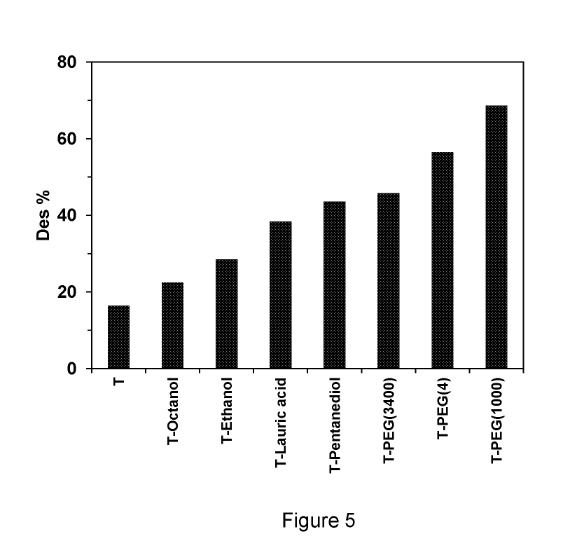

FIG. 5 depicts the efficiency of each compound. PEG yielded the best results, which confirms that the multiplicity of hydrogen bonds that a compound can form is a key factor in the desorption process. PEG₃₄₀₀ because of its lower solubility (compared to PEG₁₀₀₀), was less efficient than PEG₂₀₀ or PEG_{1000.} Indeed, the solubility of PEG in toluene decreases as the molecular weight increases. This shows that use of a soluble compound as adjuvant is ideal. A person skilled in the art would be able to vary chain length to determine the most suitable adjuvant, whether PEG or fatty acids.

### Example 6

Experiments were conducted using PEG₁₀₀₀ and EC in toluene saturated with water (0.032% determined by volumetric Karl Fischer titrator). The same experimental procedure described above was used except that toluene (T) was replaced by water saturated toluene (WT). For successive desorption, after the first desorption experiment, the residue was recovered by centrifugation, dried in an oven overnight and re-dispersed in the solvent. The mixture was stirred again for 12 h (without adding competitive adsorbent) at ambient temperature, centrifuged and the desorbed asphaltenes determined spectrophotometrically in the supernatant.

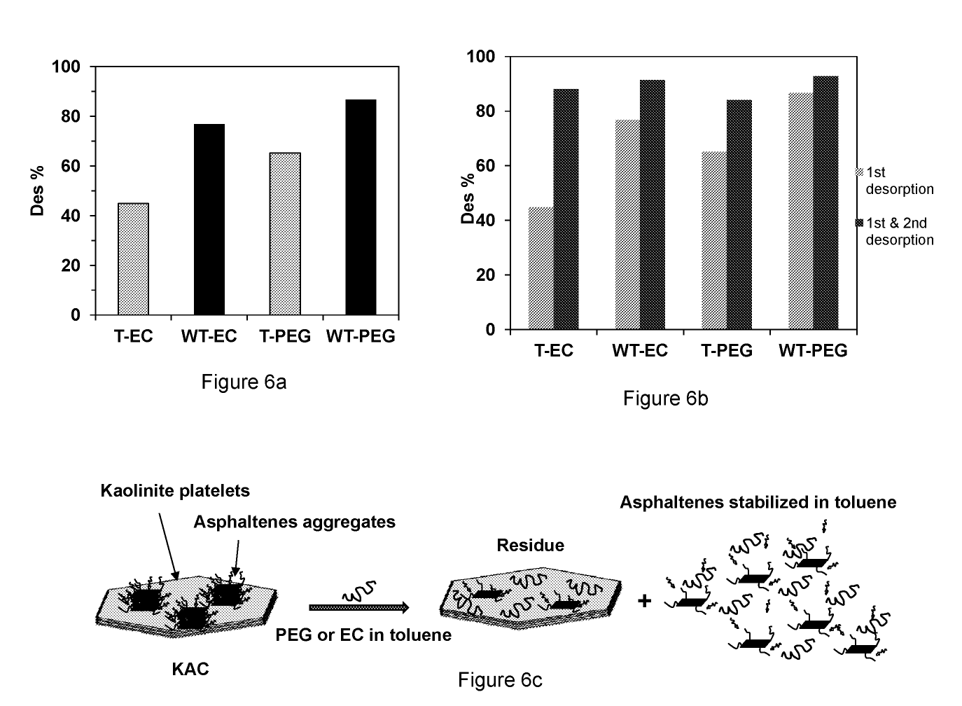

FIG. 6a shows that the presence of water increased desorption. The greatest increase was observed with EC. Desorption increased from 45% to 77% when toluene is saturated with water. With PEG, desorption increased from 65% to almost 90%. The effect of water seemed to confirm the hypothesis of the role played by hydrogen bonds in desorption of asphaltenes. FIG. 6b shows the result when the residue (with bound adsorbent or adjuvant) is added back to toluene or water-saturated toluene to perform a second desorption. The asphaltenes were almost completely desorbed by PEG or EC after two consecutive desorptions (FIG. 6b). These results showed that the process described herein (the mechanism is depicted in FIG. 6c) is very efficient for the removal of the heavy fraction of bitumen, a fraction which is typically very difficult to recover during bitumen extraction.

### Example 7

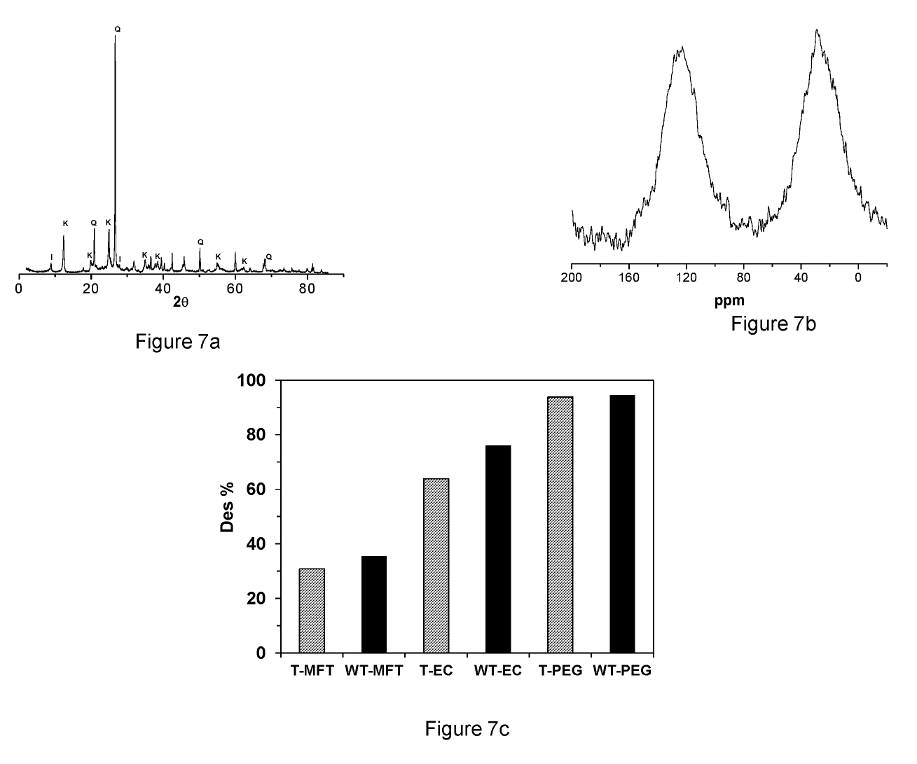

The processes that had been successfully tested on a synthetic composite (KAC) were applied on Mature Fine Tails (MFTs). MFTs are highly polluting residues resulting from the extraction of bitumen from oil sands. They consist of the fine fraction of oil sands (clay fraction, essentially kaolinite and illite) covered by bitumen. MFTs were provided by Syncrude Canada Ltd. They are characterized by their ability to form stable suspensions in water. In these experiments, MFTs had the following composition: bitumen: 1-2 wt %, naphtha <0.1 wt %, clay 30-60 wt % and the remaining was water. The MFTs paste was first dried in open air and then in an oven at 60° C. for 24 hours. The resulting solid was ground in a mortar and passed completely through a sieve of mesh diameter 300 μm. This powder contains mainly kaolinite, illite and quartz (FIG. 7a). The presence of bitumen was confirmed by the two broad bands of aromatics and aliphatics on the ¹³C NMR spectrum (FIG. 7b).

The bitumen content of the MFT was determined by a quantification method based on the results obtained on desorption of C5 asphaltenes from kaolinite particles. In practice, 2 g of MFT and 0.5 g of PEG was introduced into an extraction thimble (previously weighed) in a Soxhlet extraction apparatus. The solid mixture was continuously extracted with toluene (or chloroform) for 3 days. The thimble containing the extraction residue was then dried in oven at 60° C. for 10 hours and weighed. The mass percentage of the adsorbed PEG (3-4%) in the residue was determined by performing a TGA analysis of the composite. The MFT mass difference before and after extraction was 14% and represents the mass percentage of bitumen in MFT used for these series of experiments. A simple extraction performed without PEG yielded only 5% of bitumen.

To assess bitumen desorption, 50 mg MFT was dispersed in 5 mL toluene or water saturated toluene (VVT). 10 mg of competitive adsorbent (20%) was then added, and the mixture was stirred for 5 hours at room temperature. After centrifugation, the UV-vis spectra of the supernatants were recorded. Only EC and PEG were used as competitive adsorbents. The results in FIG. 7c once again confirmed that PEG was the most effective compound, and allows almost full desorption in a single step. The presence of water in toluene increases desorption, especially in the case of EC. These results confirm those obtained with the composite model (KAC) and show that asphaltenes are the most difficult component in bitumen to be desorbed.

### Example 8

Other solvents were tested on MFTs. The structures of these solvents are shown below. Anisole (99%) was purchased from Fluka, Methyl tert-butyl ether (99.8%) was purchased from Sigma-Aldrich and 2-Methyl furan (99%) was purchased from Aldrich.

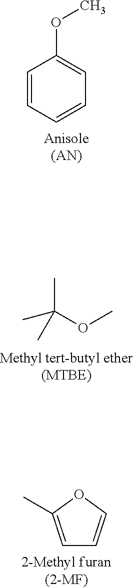

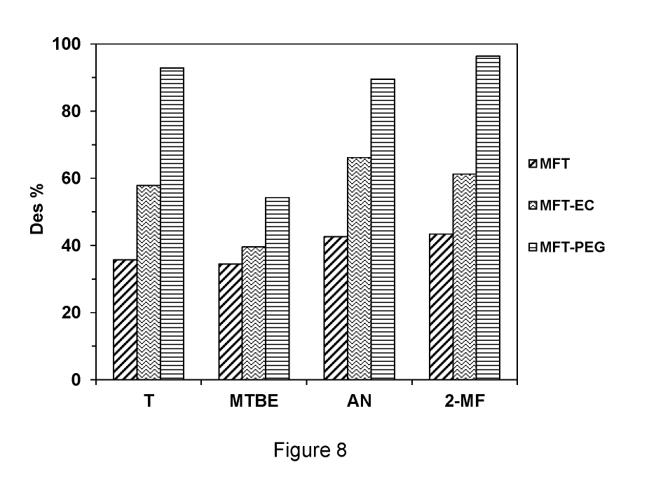

The experimental procedure was identical to that described for Example 7, except that toluene was replaced with either anisole, MTBE or 2-methyl furan. The results obtained are presented in FIG. 8. Anisole and 2-Methyl furan showed similar results to those obtained with toluene. MTBE was less efficient, presumably due to its poor ability to solubilize the competitive adsorbents.

1. A method for extracting bitumen from mature fine tails (MFTs) particles, the method comprising:
adding a compound capable of binding to solid MFTs to a dispersion of MFTs in a non-aqueous solvent, wherein the same compound is able to stabilize the bitumen fraction within the MFTs in a non-aqueous solvent;
allowing the compound to interact with the MFTs in a non-aqueous solvent for a period of time sufficient to allow the compound to adsorb onto the MFTs particles;
allowing the compound to interact with the MFTs in suspension in an non-aqueous solvent for a period of time sufficient to allow the compound to stabilize the bituminous fractions in the organic solvent;
separating the residual solid fraction of MFTs;
separating the bitumen; and
recovering the solvent.

2. The method of claim 1, wherein the extraction is carried out in toluene.

3. The method of claim 1, wherein the MFTs comprises asphaltenes.

4. The method of claim 1, further comprising agitating the compound with the MFTs to improve adsorption of the compound onto the bitumen.

5. The method of claim 1, further comprising:
recovering the bitumen following separation;
adding a second compound to the recovered bitumen for a time sufficient to allow the second compound to adhere to the clay particles in the recovered bitumen;
separating a second residual solid fraction of MFTs;
recovering additional bitumen; and
recovering the solvent.

6. A process for separating bitumen from clay, the process comprising:
dispersing a composition comprising bitumen and clay in an organic solvent;
adding an adsorbent capable of adsorbing onto the clay;
agitating the mixture; and
separating a residue comprising a clay-adsorbent from the bitumen released into the organic solvent.

7. The process of claim 6, wherein the adsorbent is cellulose, ethylcellulose, methylcellulose, or polyethylene glycol.

8. The process of claim 6, wherein the solvent is toluene, anisole, methyl tert-butyl ether, or 2-methyl furan.

9. The process of claim 6, wherein the composition of bitumen and clay is mature fine tailings.

10. A method to determine the amount of bitumen in MFTs, the method comprising:
adding an adsorbent to dry MFTs;
extracting the mixture of adsorbent and MFTs using an organic solvent;
drying the extracted residue; and
determining the amount of extracted bitumen by measuring the mass balance of MFTs before and after extraction.

11. The method of claim 10, wherein the adsorbent is PEG and the residual PEG adsorbed onto extracted MFTs is determined by TGA analysis of the residue.

12. The method of claim 10, wherein the adsorbent is EC or other suitable adsorbent.

13. The method of claim 10, wherein the organic solvent is toluene, chloroform, or other suitable aromatic solvent.
